(19) United States
(12) Patent Application Publication
(10) Pub. No.: US 2016/0023016 A1
(43) Pub. Date: Jan. 28, 2016

(54) ELETROMAGNETIC CORTICAL STIMULATION DEVICE

(71) Applicants: Giorgio BONMASSAR, Lexington, MA (US); Yoshio OKADA, Boston, MA (US); THE GENERAL HOSPITAL CORPORATION, Boston, MA (US)

(72) Inventors: Giorgio Bonmassar, Lexington, MA (US); Yoshio Okada, Boston, MA (US)

(21) Appl. No.: 14/774,493

(22) PCT Filed: Mar. 10, 2014
(86) PCT No.: PCT/US14/22363

§ 371 Date: Sep. 10, 2015

Related U.S. Application Data
(60) Provisional application No. 61/775,785, filed on Mar. 11, 2013.

(51) Int. Cl. A61N 2/02; A61N 1/05; A61N 2/00

(52) U.S. Cl. CPC A61N 2/02; A61N 2/006; A61N 1/0553

(57) ABSTRACT

An electromagnetic cortical stimulation array structured to stimulate cortical regions during a presurgical localization of eloquent areas. The device includes array of miniature elongated magnetic coils (optionally overlaid on an array of electrodes) oriented substantially parallel to one another and structured to be inserted under the dura during an open cranial neurosurgery. Each coil and electrode is activated (optionally, selectively) to subdurally stimulate different regions of the cortex to determine functions of each cortical area.

### CROSS-REFERENCE TO RELATED APPLICATIONS

This international patent application claims priority from and benefit of the U.S. Provisional Patent Application No. 61/775,785 filed on Mar. 11, 2013 and titled “Electromagnetic Cortical Stimulation Device”. The disclosure of the above-identified provisional application is incorporated by reference herein in its entirety.

### TECHNICAL FIELD

The present invention relates to stimulation of a spinal cord and, in particular, to an implantable device having an array of selectively operable magnetic coils wrapped around the biological tissue containing neurons to be stimulated.

### BACKGROUND

Problems with spinal cord such as spinal cord injury (SCI), for example, command serious attention around the globe. In the USA alone, there are about 250,000 patients and about 11,000 new cases each year. Extensive functional recovery is sought when the injury is partial and does not sever the cord completely. The spinal cord anatomy is well suited for epi- or subdural stimulations since the white matter occupies the superficial 25-50%. Stimulation of the spinal cord with electrical current is becoming a useful treatment modality for patients with spinal cord injury (SCI) when it is combined with molecular biological, pharmaceutical and surgical techniques. Furthermore, functional electrical stimulation (FES) is also performed using a spinal cord stimulator to control chronic pain. FES may improve certain motor disorders such as spinal spasticity or augment standing or ambulation by implanting the stimulation electrodes in the epidural lumbar region of the spinal cord. FES in the spinal cord includes stimulating electrodes, conductive leads to carry the electrical pulse and electrical pulse generator, which is usually implanted in the abdominal area or gluteal region of the patient.

Conventionally, spinal neurons are activated by applying voltage or current to one or more electrodes in an array of electrodes placed on the dura of the spinal cord. The pulse generator can be programmed using RF or magnetic fields, so that the operator can select the electrode stimulation pair or pairs, pulse timing and amplitude. The generator can either be a voltage or a current generator delivering either voltages or milliamperes to the electrodes. The timing of the pulse can be controlled and a single pulse usually varies between about 100 and about 500 microseconds with a period in between pulses from about 5 to about 50 ms. Both are controlled by the patient sensation and sensory/motor area that is being treated.

In clinical applications, the electrical stimulation of the spinal cord involves laminotomy and insertion of an electrode array on a strip through a hole in the lamina into the epidural space. Two adjacent electrodes are often used as a pair of bipolar electrodes (12) to provide focal stimulation of the spinal neurons near the lumbar-sacral bulge. The utility of an epidural array has also been demonstrated. Other types of invasive electrical stimulation methods are being tested, that include flexible electrode array that can be wrapped around the spinal cord, prong electrodes made of microfabrication techniques, or microwires that can be inserted into the white and gray matters.

Epi- and subdural electrodes have the problems of effective and consistent stimulation, especially in chronic preparations (approximately corresponding to implantations with terms longer than about 4 weeks). Another serious problem is immune reaction of the tissue to the metal used for the electrodes. Scar tissue may develop around electrodes in chronic use. This increases the electrical impedance and reduces the effectiveness of electrical stimulation and in some cases cord compression. Most of these problems are present for subdural stimulations, even though the electrodes are closer to the cord. Exposed electrodes produce reactive tissues in chronic preparations, leading to increase in electrical impedance. Cerebrospinal fluid (CSF) shunts the volume current produced by the electrode(s). Electrical stimulation can also produce excessive tissue heating.

Intraspinal implantable electrodes have been proposed to overcome the limitation of spatial resolution, but they suffer from several serious shortcomings. The important problem for clinical application is tissue damage caused by the flexing motion of the spinal cord during the movement of a patient that leads to dislodging of the implanted electrodes or wire leads; or damage to the spinal cord. “Floating electrodes” and flexible electrodes made of microelectrodes and wires are being developed to address this problem, but they are still in experimental stages.

There remains a need, therefore, for a device and method for stimulation of the spinal cord tissue that is devoid of the shortcomings presented by cortical stimulation with electrical pulses.

### SUMMARY

Embodiments of the present invention provide a device for stimulating a biological tissue. The device includes an element capable of coexisting with living tissues and organisms without causing any harm, which is substantially neutral with respect to interaction with a biological tissue. Such element (that alternatively may be referred to as a biocompatible element) is structured to have a sheet of electrically non-conductive material, first and second surfaces, and first and second ends. The biocompatible element contains a plurality of substantially equal segments extending along an axis between the first and second ends, and the immediately neighboring segments adjoin one another along the axis. The device additionally includes microcoil systems juxtaposed with the biocompatible element such as (i) to be electrically insulated from the first and second surfaces, and (ii) to form an array of microcoil systems that is oriented substantially transversely to the axis. Generally, the microcoil systems are substantially evenly spaced in the array of the microcoil systems. Each segment of the element is associated with a microcoil system that extends between the first and second ends. The element is structured to be pliable to enable bending of the first sheet to define a cylindrical surface.

In a specific case, the biocompatible element includes a biocompatible implant structured for subdural placement. Alternatively or in addition, a device may include a biocompatible coating covering at least one of the first and second surfaces and forming at least a portion of an electrical insulating barrier that covers and/or encases the microcoil systems.

In one embodiment, the device may further include a power coupling element or extension dimensioned to connect the microcoil systems to a power source to drive the microcoil systems, as a result of which connection the microcoil systems are operable to produce magnetic fields suitable for performing cortical stimulation. More specifically, such embodiment may additionally include a stimulator electronic circuitry configured to control a delivery of power from the power source to the microcoil systems to deliver electrical current to a selected combination of the microcoil systems and according to a preselected pattern, in order to perform cortical stimulation. An embodiment may additionally include a set of bipolar electrodes a number of which equals a number of the microcoil systems. Here, each bipolar electrode is disposed along the axis on one of the first and second surfaces such as to overlap with a corresponding microcoil system at the ends of the microcoil system. The bipolar electrodes and microcoil systems are electrically insulated from each other. A pole of a bipolar electrode from the set may include a substantially circularly-shaped electrically-conductive patch which, in a specific case, may be patterned as a comb.

Alternatively or in addition, a microcoil system of the embodiment o the device may include a plurality of microcoil elements disposed one over another in layers (wherein first and second immediately neighboring layer are separated from one another by a layer of a microcoil-encasing electrically non-conductive material). Each of the microcoil elements has a corresponding via or passage, and the microcoil elements are electrically connected to each other through corresponding vias. First and second microcoil elements may be dimensioned in such a fashion as to make their foot-prints on the first surface of the implant to be substantially co-extensive. Alternatively or in addition, the microcoil system may include a ferrite-based core extending across the layers of microcoil elements. The micro-coil-encasing electrically non-conductive material may include biocompatible material.

Embodiments of the invention additionally provide a spinal cord stimulation device that includes (i) a power source enabled to produce electric pulses; and (ii) an element structured as a sheet containing an embedded array of microcoil systems that are electrically insulated from ambient medium. The microcoil systems are structured to receive the electric pulses to generate respectively corresponding magnetic fields directed to induce corresponding electrical fields adjacent to the implant sheet and suitable to perform subdural cortical stimulation. The sheet is bendable to form a substantially cylindrical surface to direct the vectors of the respectively corresponding magnetic fields towards an axis associated with the cylindrical surface. In a specific implementation, element includes a biocompatible implant sheet.

The element may be bendable such as to form a substantially cylindrical surface. When so bended, vectors of the magnetic fields generated by the microcoil system of the element are directed towards an axis corresponding to the cylindrical surface. Alternatively or in addition, a microcoil system from the array includes a multilayer structure containing layers with individual microcoils alternating with layers of electrically insulating material, the individual microcoils being electrically connected to each other through vias or passages formed across the layers of electrically insulating material. Alternatively or in addition, the stimulation device may further include an array of bipolar electrodes overlapped with the array of microcoil systems such that poles of each individual bipolar electrode overlap with extreme points of a respectively corresponding microcoil system. The bipolar electrodes are operably connected with the power source to receive said electrical pulses. Moreover, an embodiment of the device may additionally include a controlling circuitry (or controller, for short) configured to selectively govern or control the delivery of the electrical pulses to the microcoil systems to effectuate cortical stimulation in a pre-determined spatial pattern.

### BRIEF DESCRIPTION OF THE DRAWINGS

The invention will be more fully understood by referring to the following Detailed Description of Specific Embodiments in conjunction with the Drawings, of which:

FIG. 1 is a diagram schematically presenting a spinal cord stimulation system;

FIG. 2A is a depiction of an embodiment of the invention juxtaposed with a carrier shaped to define a cylindrical surface;

FIG. 2B is a diagram illustrating spatial orientation of an array of coils of the embodiment of FIG. 2A with respect to target tissue;

FIGS. 3A and 3B provide illustrations to coil structures for use with an embodiment of the invention;

FIG. 4A illustrates a grid of bipolar electrodes disposed on a carrier shaped to define a cylindrical surface;

FIG. 4B is a depiction of distribution of primary electric field formed by a bipolar electrode placed in proximity to the spinal cord;

FIG. 5 is a diagram illustrating a portion of an alternative embodiment of the invention including an array of individual elements;

FIG. 6A is a detailed depiction of an individual element of the embodiment of FIG. 5;

FIG. 6B is a diagram presenting, in a cross-sectional view, layered structure of the individual element of FIG. 6A;

FIG. 6C is a series of diagrams representing coils at immediately neighboring layers of the layered structure of FIG. 6B;

FIG. 7 provides illustration of orientation of the layered structure of FIG. 6B in relation to the target tissue;

FIG. 8A is a diagram depicting an embodiment of the invention containing two subdurally insertable parts that, in cooperation, substantially encase the spinal cord along its length;

FIG. 8B is a diagram corresponding to the diagram of FIG. 8A, in which the cross-sectional view of the embodiment is overlapped with a cross-sectional view of the spinal cord.

FIGS. 9A, 9B, 9C provide depictions of distribution of primary electric field formed by an embodiment of the invention in proximity to the spinal cord; a voxel of tissue penetrated by the electric field; and an equivalent electrical circuit for such voxel, respectively.

FIG. 10 is a graph representing point spread functions characterizing the activation of neurons with embodiments of the invention in comparison to that characterizing the activation of spinal cord neurons with a single bipolar electrode.

FIG. 11 is a graph characterizing accuracy of activation targeting of spinal cord neurons with embodiments of the present invention as compared with that associated with a single electron activation targeting.

FIG. 12 is a schematic representation of electronic circuitry of the stimulation system to drive a single (left or right) EM array of the embodiment of FIGS. 8A, 8B.

### DETAILED DESCRIPTION

An electromagnetic cortical stimulation array (EMCSA) device is described that is structured to stimulate cortical regions during a presurgical localization of eloquent areas. It includes, in relevant part, an array of miniature elongated magnetic coils (optionally overlaid on an array of electrodes) oriented substantially parallel to one another and structured to be inserted under the dura during an open cranial neurosurgery. Each coil and electrode is activated (optionally, selectively) to subdurally stimulate different regions of the spinal cord tracks to determine functions of each fiber track which, to the knowledge of the inventors, has not been done yet. The device provides important advances in techniques for stimulation of eloquent areas since the EMCSA increases efficiency in stimulating deep and superficial spinal cord tracks.

Conventionally, the functional electrical stimulation has been carried out using commercially available electrode arrays. The electrodes depolarize neurons and activate the underlying cortical area. The electrodes by their fundamental nature tend to preferentially stimulate superficial than deep fibers, despite the fiber directionality. Thus, the stimulation is incomplete. In contradistinction, the use of a magnetic coil array by itself facilitates preferential activation over a certain direction, with the current having a mirror-image shape of the current in the coil. (see, for example, Bonmassar G. et al., in Microscopic magnetic stimulation of neural tissue, Nature Communications, pp. 1-10, 26 Jun. 2012). Fibers can be activated or not depending on the magnetic field direction. The combination of the magnetic coil array and electrodes, therefore, enables the modality for more efficient stimulation of both deep and superficial. Spinal cord fibers will be targeted to stimulate using this array, by employing the so-called beamformer technique, which is sometimes called synthetic aperture magnetometry (SAM) in the field of magnetoencephalography (MEG). The beamformer technique was developed originally in the field of radio astronomy to beam the radio signal from an array of antennas onto a target in the universe. The beamformer technique has been developed in the field of MEG in order to localize activities in the brain with a high spatial precision. Each MEG sensor has a sensitivity function (so-called lead field). By combining the sensitivity function of more than one channel, the beamformer constructs a synthesized sensitivity function which is much sharper than the sensitivity function of individual sensors. This synthesized sensitivity function can be beamed to the desired target region to detect brain activity in the region. The process is reciprocal and the same technique can be used to stimulate desired target regions in the spinal cord.

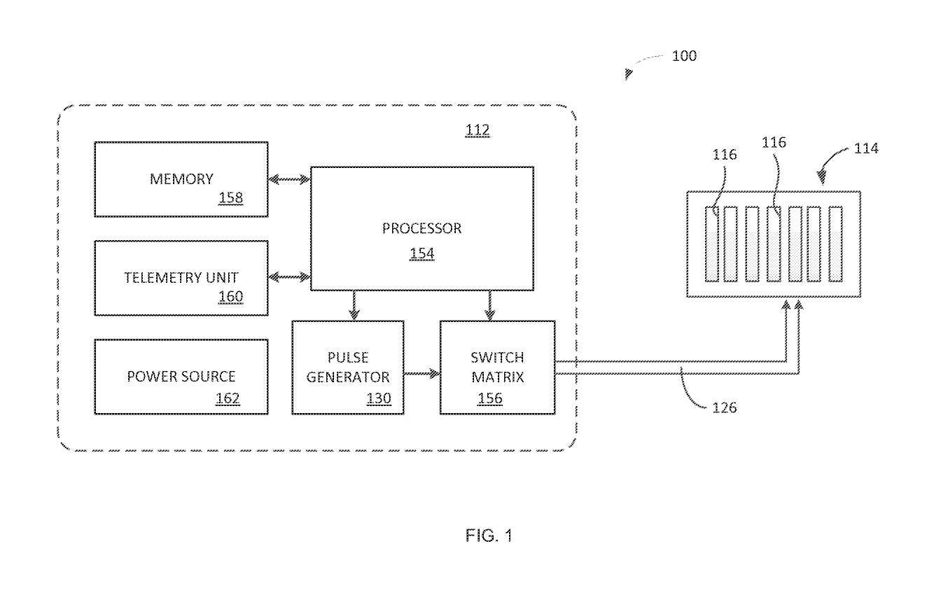

Referring initially to FIG. 1, a spinal cord stimulation system 100 includes a stimulator 112 coupled to an implantable device 114 (referred to hereinafter as an ‘implant’ and shown in FIG. 1 schematically and without reference to any particular structure) that contains an array of electromagnetic microcoils 116. The stimulator 112 includes a pulse generator 130 that generates electrical pulses for delivery to a targeted stimulation site in a spinal cord (not shown) via the implant 114 that embraces the spinal cord as a result of installation, as discussed below. The electrical pulses cause the microcoils 116 to produce magnetic fields with magnetic field vectors that are directed into a spinal cord substantially transversely to surfaces defined by the microcoils 116. These magnetic fields, in turn, induce electrical currents in the spinal cord tissue to excite the neurons therein.

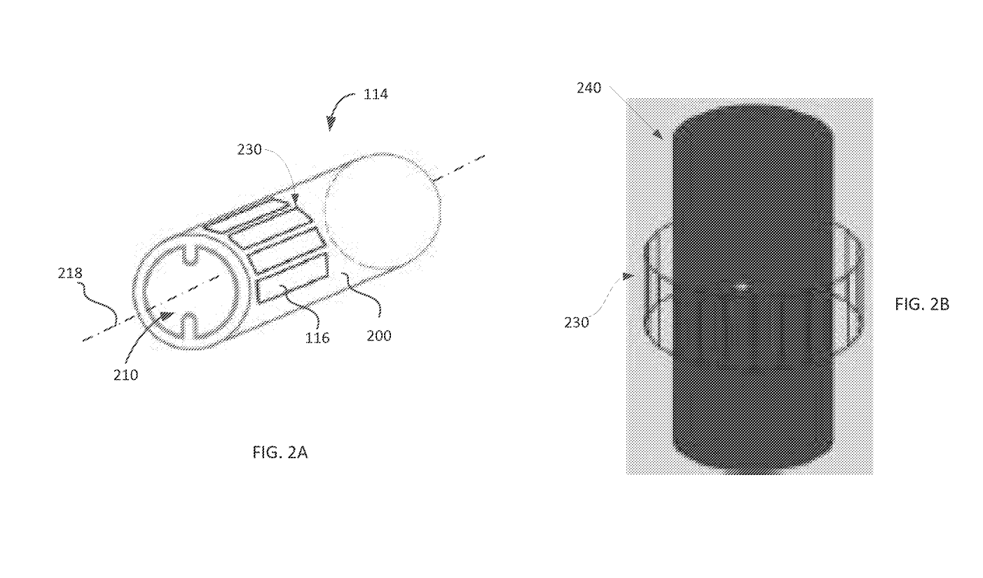

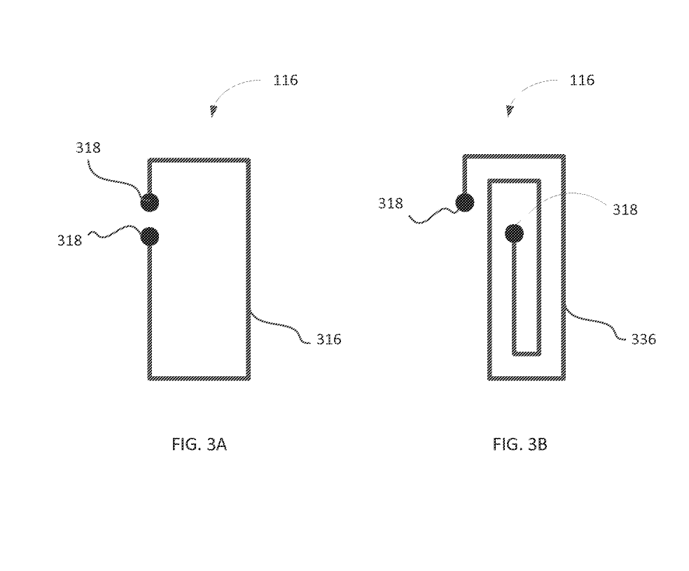

In reference to FIG. 2A and in further reference to FIG. 1, the implant 114 is structured to define, when installed and/or assembled, an elongated hollow (tubular) body 200 having an internal volume 210 extended along the axis 218. In a specific case, the tubular body 200 may be substantially cylindrical, for example about 3.5 in diameter. The implant 114 is electrically coupled to the stimulator 112 via a coupling or coupling extension 126 (that may include at least one electrical lead or a wireless coupling). Multiple spaced-apart microcoils 116 form at least one array 230 of microcoils which, like a belt, encircles after the installation the axis 218 and the target biological tissue 240 with neurons around which the body 200 with the array 230 is disposed. In the simplest implementation, in reference to FIG. 3A, a microcoil 116 may be shaped as an open ring or loop 316 of a metallic trace having electrical terminals 318. Alternatively, FIG. 3B, the microcoil 116 may be structured as a spiral 336 including multiple loops of metallic traces. The implant 114 also includes at least one electrical conducting member (not shown) connected to the pads 318 and a ground layer (not shown) juxtaposed with the array 230 and a surface of the body 200 on which the array 230 is disposed. Each of the microcoils 116 is coupled to one of the electrical conductors and to the ground layer.

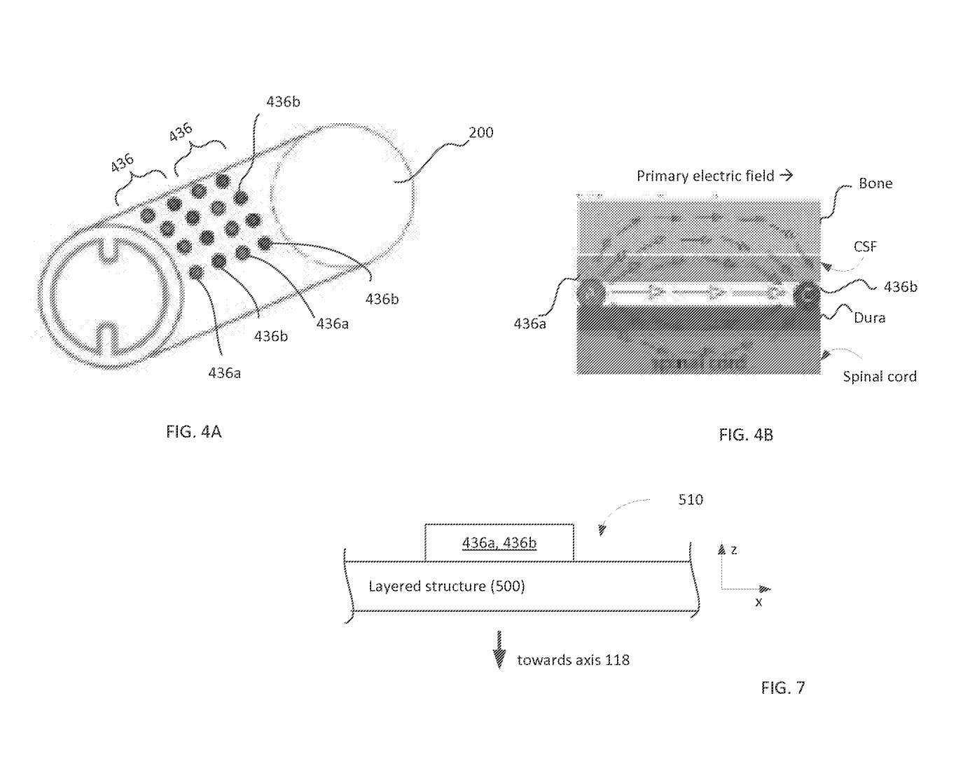

The implant device containing the array 230 of microcoils 116 can optionally be complemented with a corresponding array of electrodes or electrode grid. FIG. 4A is a diagram depicting the disposition of only the grid 430 of the bipolar electrodes 436 on a surface of the body 200. Each of the bipolar electrodes has poles 436a, 436b and is shown in FIG. 4 without any particular structural detail. By itself, the epidural use of a grid of electrodes suffers from the problem of poor spatial specificity in stimulating target spinal neurons. The dura has poor electrical conductivity relative to the CSF. As a result, there exists a large drop in electrical potential across the dural membrane (as shown schematically in FIG. 4B), since the drop is inversely proportional to the conductivity of the membrane. The CSF, due to its high conductivity, smears or spread the volume current emerging from the dura. The electric field of each epidural electrode within the spinal cord is wide spread and greatly attenuated, with the result of substantially poor control in stimulating various neuronal structures in the spinal cord. Bipolar stimulation provides more focal stimulation, but it is still quite broad (as shown schematically in FIG. 4B). The electric field created by a bipolar electrode has radial and longitudinal components, as a result of which the density of the electric field is not uniform. The electric field formed by the bipolar electrode effectively depolarizes neurons in the spinal cord near the cathode and hyperpolarizes the neurons in the vicinity of the anode. Electrodes could activate soma/dendrites of the neurons in the grey matter as well as axons in the white matter.

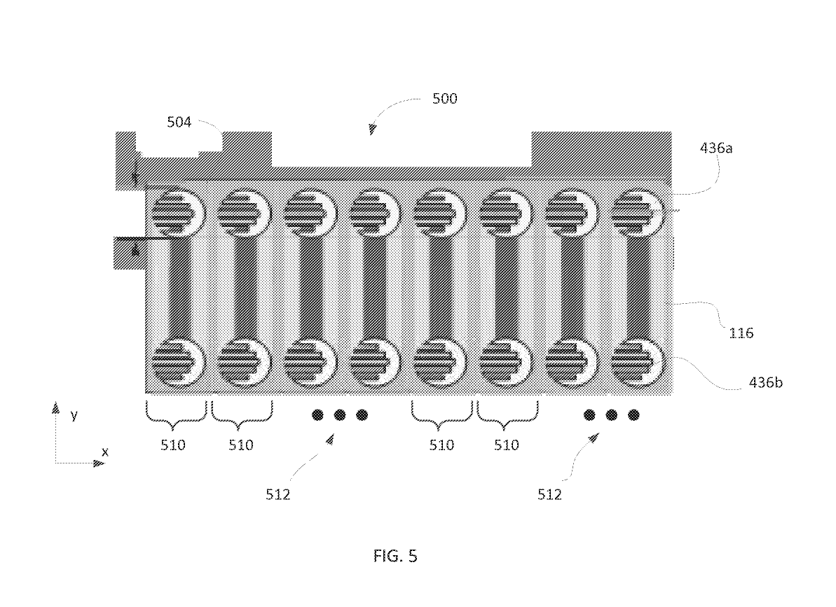

FIG. 5 depicts an example of the embodiment that employs a specifically spatially coordinated with one another the array 230 of FIG. 2A and the grid 430 of FIG. 4A. As shown, the embodiment of FIG. 5 is flattened along the xy-plane. Here, the array 230 and the grid 430 aggregately form an electromagnetic (EM) array 500 supported by a flexible carrier. The array 500 includes an array of sequentially abutting and linearly connected to each other individual EM elements 510 (interchangeably referred to herein as EM channels), each of which is structured to include the bipolar electrode 436 and the microcoil 116, which as juxtaposed to one another along the z-axis. The EM array 500 is structured to be housed in and supported by a carrier 504 such as a layer of conformable plastic (for example, a layer of Teflon between about 0.3 and 1.1 mm thick) that can be bent to form at least a portion of the tubular body 200. As the carrier is flexible, the array 500 is enabled to pliably fit around the target tissue (such as a spinal cord; for example about 3.5 mm in diameter). In a specific case, the EM array 500 may include between 14 and 24 individual EM elements or channels 510. Ellipses 512 of FIG. 5 indicate the repetition of the structure of the array 500.

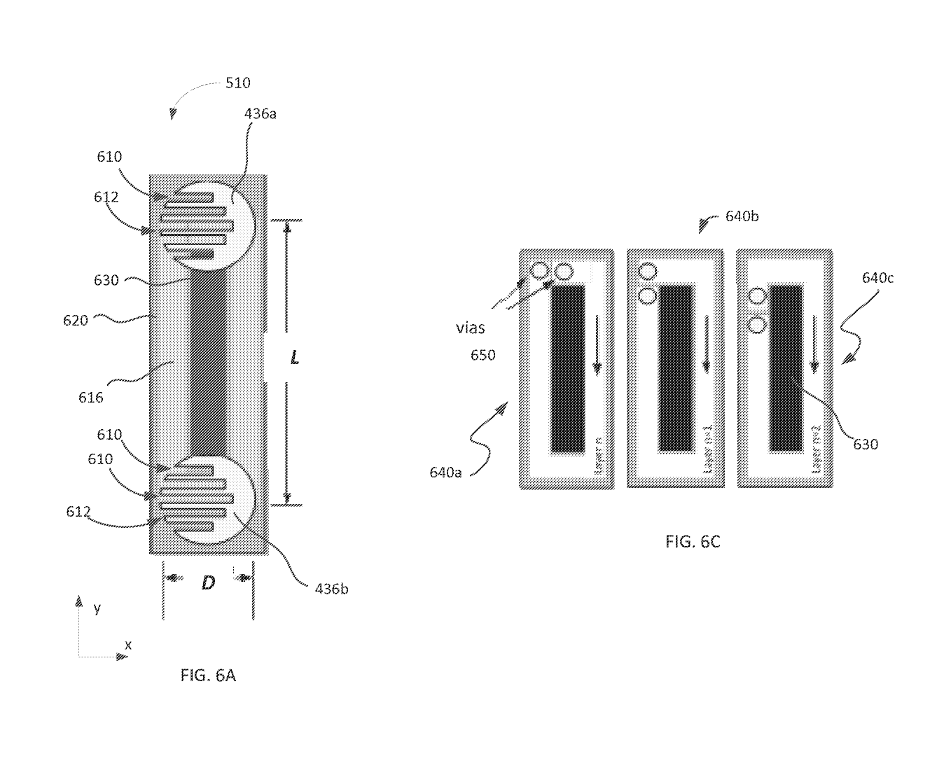

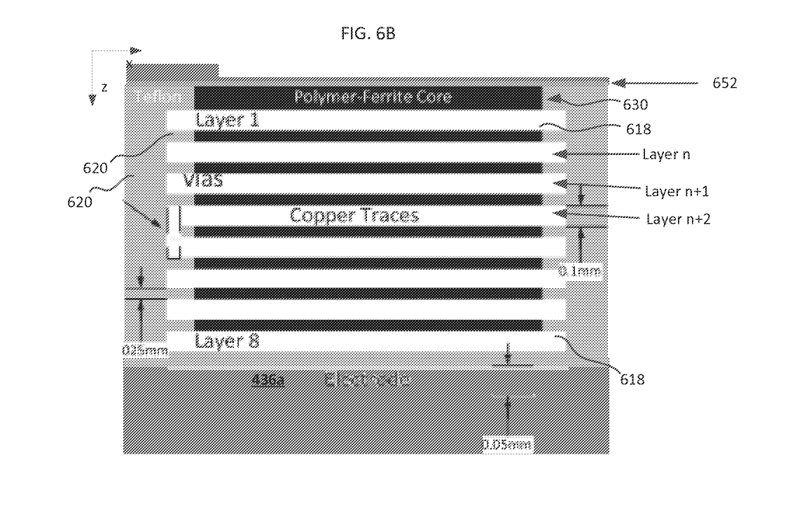

In further reference to diagrams of FIGS. 6A, 6B, 6C, details of a single element 510 of the EM array 500 of the embodiment of the invention are discussed. As shown in FIG. 6A, the electrodes 436a, 436b include substantially circular patches (for example, having diameters of about 0.4 mm and thickness of about 25 microns) that are fabricated on a plastic substrate using known printing, lithographic, or thin-film deposition methods. In a specific case, the electrodes 436a, 436b have complex shapes including slits 610 that define a multiplicity of thin-film fingers or comb 612 in each of the substantially circular patches of the electrodes 436a, 436b. Such structuring of the electrodes 436a, 436b facilitates the reduction or prevents the formation of Foucault currents (eddy currents) in the electrodes 436a, 436b during the formation of the magnetic field by the coil system 616.

FIG. 6B illustrates a cross-section of the element 510 with the xz-plane at a point chosen across the electrode patch 436a. Each coil system 616 has a plurality of layered electrically-conductive loops 618 (for example, eight layers each having a loop 618 structured similarly to the loop of the coil 316 of FIG. 3A, about 100 micron thick). The layered loops 618 are separated by layers of the housing plastic material 620 (for example, Teflon). A single loop of the electrically-conductive coil system 616 (which may be made of copper, for example) is about 0.4 mm (D) by about 1.2 mm (L). The coil system 616 optionally has a ferrite-impregnated polymer core 630 (having relative permeability μᵣ of, for example, greater than 600) to amplify the magnetic field generated by the coil system 616. Each coil system 616 is connected by a set of electrically-conductive members (traces) with pin-connectors (not shown) disposed, in one implementation, at the edge of the array 500. The components of the coil system 616 are molded into, laminated, or encased in the housing material 620. As shown, a thin-film patch 436a of the bipolar electrode 436 of FIG. 4 is disposed on the outer surface of the housing material 620.

FIG. 6C depicts, for illustration purposes only, three consecutive layers 640a, 640b, 640c (layer n, layer n+1, and layer n+2) of the coil system 616 of the embodiment 510, each having respectively corresponding vias 650 through which the individual coil elements 618 are electrically connected to one another across the layers (as shown, along the z-axis) to form chosen configuration of electrical traces.

In reference to FIG. 7, and referring again to FIGS. 5 and 6B, upon implantation of the implant 114 (carrying the array 230 or the array 500) to the spinal cord, the microcoils of the embodiment are positioned in close proximity to a target stimulation site for delivery of magnetic field pulses, while the grid of bipolar electrodes is separated from the tissue by the layered structure carrying the microcoils. (Alternatively, the EM array 510 may be oriented with the layer of electrodes 436a, 436 facing the target tissue and with the layered structure corresponding to the coil system 616 being separated from the target tissue by the layer of electrodes, although such orientation is not the most efficient in practice). Additionally, the microcoil system 616 is electrically isolated from the target tissue and there is no interface therebetween. In a simplified embodiment employing only the array of coils adjacent to the body 200 of the implant 114 (such as, for example, the embodiment of FIG. 2), the layer carrying the microcoil(s) is installed to face the target tissue directly. In this case, the only portion of the implant 114 in direct contact with the spinal cord tissue is a dielectric sheath, or coating 620.

In a specific implementation, and in further reference to FIG. 6B, at least one of the outer surfaces of the sheet of material forming the body of the implant may be additionally covered with a biocompatible material such as parylene. In this case, the portion of the implant 114 in direct contact with the spinal cord tissue is a layer of parylene.

The use of a biocompatible polymeric material and, in particular, parylene, for the coating disposed on an outer surface of the implant 114 gives the implant numerous beneficial attributes. Parylene has a low coefficient of friction (e.g., 0.025) such that the implant 114 can be inserted in contact with dura with minimal damage to adjacent tissue. Parylene has a low permeability to moisture and gases (for example, about 0.01% in water), thereby providing stable dielectric properties for the implant 114 over an extended period of time, which is of high importance for spinal cord implants. Still further, parylene exhibits fungus and bacteria resistance, thereby minimizing the likelihood of an immune response. Furthermore, parylene exhibits high tensile and yield strength (for example, 65,000/6,300 psi), thereby reducing the potential for the coating 36 to be stripped when the implant 114 is inserted next to the spinal cord. Further yet, parylene exhibits increased radiation resistance which is beneficial for the sterilization of the implant 114. Finally, as previously mentioned, parylene has a high dielectric strength (for example, 7,000 V/mil@ 1 mil), thereby providing an effective electrical insulation barrier between the implant 14 and the surrounding brain tissue.

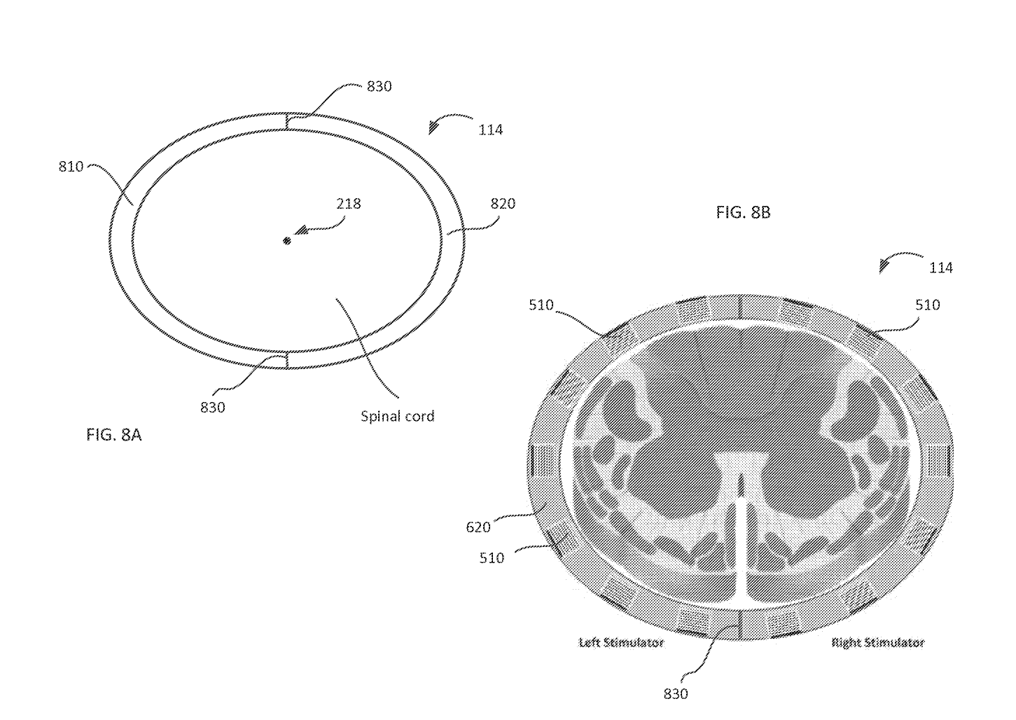

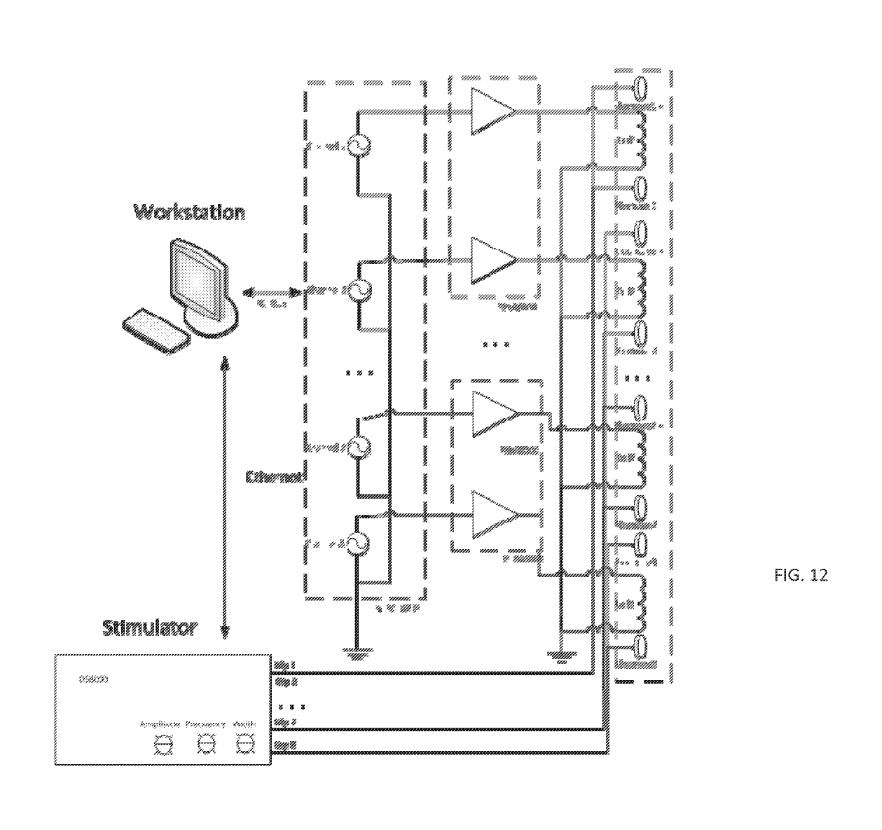

The implant 114 is designed to be placed in cooperation with the spinal cord and be wrapped around the dura. To simplify the installation around the target tissue of a spinal cord, the tubular body 200 of the implant 114 may be assembled from two or even more flexible segments each of which is positioned along a portion of the spinal cord to form, aggregately, a tubular and/or cylindrical surface around the dura and encircle the dura with an array of microcoils (in a fashion similar to that depicted in FIG. 2B). When the implant includes only an array 230 of microcoils (see FIG. 2), each of the flexible segments forming the body 200 contains at least one microcoil. When the implant includes the combination of the microcoils 116 and electrodes 436 (see FIG. 5, for example), each of the flexible segments forming the body 200 is adapted to contain at least one individual EM element 510. To this end, FIG. 8A illustrates, in a cross-sectional view, a specific embodiment of the installed implant 114. The implant 114 is shown to be assembled around the spinal cord from two flexible implant segments, 810 and 820, each of which is bent to define a semi-cylindrical half of the body 200. The implant segments 810, 820 are cooperated to contact one another along their respective edge surfaces, shown in the view of FIG. 8A as lines 830, to complete the tubular body 200 of the implant. Accordingly, each of the segments 810, 820 is bent to define a substantially cylindrical surface. FIG. 8B presents a related view: an axial section of a spinal cord with the two “C” sections of the EM array 500 (denoted as left and right stimulators). The configurations of FIGS. 8A, 8B are selected to improve the ease of surgical placement of the two “C” sections by wrapping them around the exposed spinal cord. A schematic representation of electronic circuitry of the stimulation system to drive a single (left or right) portion of the EM array of FIGS. 8A, 8B is shown in FIG. 12.

The actual number and arrangement of the microcoils 116 (or coil systems 616) about the implant 114 may vary with specific design or application considerations and are considered to be within the scope of the present invention. Other design considerations, such as the geometry (e.g., size, shape, etc.) and placement of the microcoils 116, may be adjusted depending on the amount or location of neural stimulation for a particular treatment. The induced electric field is a sum of the electric fields induced by each microcoil, and therefore, by changing the driving currents of individual microcoils 116 or coil systems 616, the area of neural stimulation can be shaped and targeted.

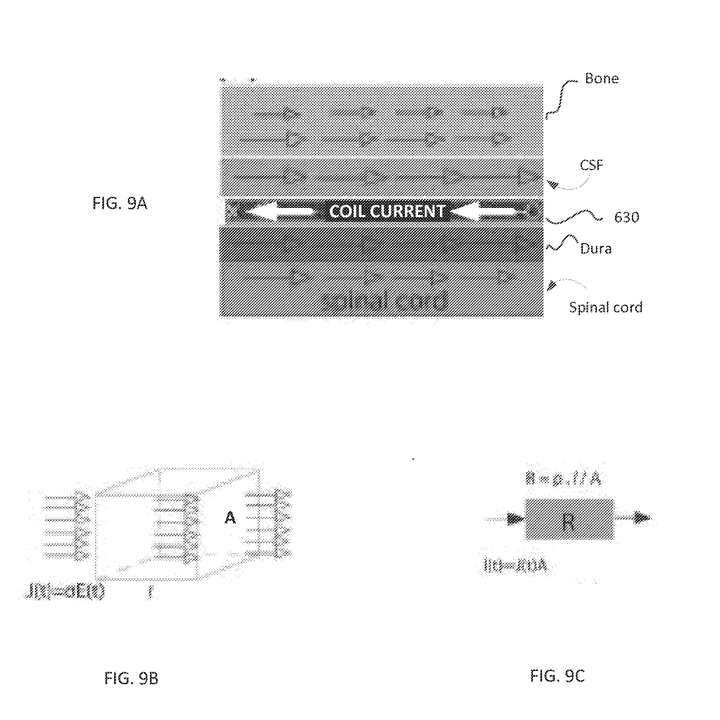

In operation, after the implant device 114 is placed subdurally, spinal neurons are stimulated by passing a short pulse of current through at least one microcoil system. This generates the magnetic field B characterized by a magnetic field vector that is transverse to a surface of the coil. The time-varying B generates the electric field E according to the Faraday's law ∇×E=(−∂B)/dt. FIG. 9A illustrates the distribution of electric field formed by a single microcoil system structured according to the embodiment of FIGS. 6B and 6C. In comparison with the spatial distribution of the electric field formed by bipolar electrode (FIG. 4B), the vector of the electric field formed by the microcoil system is nearly parallel to the plane of an individual coil of the microcoil system in different tissues adjacent to the coil (in accord with the Lenz' law). Neuronal processes such as axons parallel to the vector of so directed electric field are depolarized, but the processes transverse to the E-vector are not. Accordingly, the operation of the microcoils of the system enables preferential activation of the axons along the spinal-cortical and cortical-spinal tracts in the white matter. FIGS. 9B and 9C provide illustrations to a voxel of tissue penetrated by the electric field of FIG. 9A and an equivalent electrical circuit for such voxel, respectively.

Referring again to FIG. 1, the stimulation system 112 may further include a processor 154 to set the amplitude, pulse width, and pulse rate parameters of stimulation pulses applied to the implant 114. The processor 154 may be realized by one or more microprocessors, digital signal processors (DSPs), Application-Specific Integrated Circuits (ASIC), Field-Programmable Gate Arrays (FPGA), or other equivalent integrated or discrete logic circuitry. The stimulation system 100 may further include a switch matrix 156 to apply the stimulation pulses across selected microcoils 116 or microcoil systems 616 within a single portion of the implant 14 or within two or more implant portions. The stimulation pulses may be applied in a bipolar or multipolar arrangement, in which multiple microcoils 116 are selected for delivery of stimulation pulses, for example, across or among different microcoil pairs or groups. Alternatively, the stimulator 12 may include multiple pulse generators 130, each coupled to and controlling a given series of microcoils 116.

A tangible non-transitory computer-readable memory 158 may be provided to store instructions for execution by the processor 154 to control the pulse generator 133 and the switch matrix 156. For example, the memory 158 may be used to store programs defining different sets of stimulation parameters and microcoil combinations. Other information relating to operation of the stimulator 112 may also be stored. The memory 58 may include any form of computer-readable media such as random access memory (RAM), read only memory (ROM), electronically programmable memory (EPROM or EEPROM), flash memory, or any combination thereof.

A telemetry unit 160 supporting wireless communication between the stimulator 112 and an external programmer (not shown) may be provided. The processor 154 controls the telemetry unit 160 to receive programming information and send operational information. Programming information may be received from an external clinician programmer or an external patient programmer. The wireless telemetry unit 160 may receive and send information via radio frequency (RF) communication or proximal inductive interaction of a programmer.

A power source 162 delivers operating power to the components of the stimulator 112 including the microcoils 116. The power source 162 may include a rechargeable or non-rechargeable battery or a power generation circuit to produce the operating power. In some embodiments, battery recharging may be accomplished through proximal inductive interaction between an external charger and an inductive charging coil within the stimulator 112. In other embodiments, operating power may be derived by transcutaneous inductive power generation, e.g., without a battery.

### Advantages of the Proposed Solution.

(1) Reliability and Stability of Coil.

Each microcoil of the device is completely encased in a biocompatible plastic material. This protects the coils unlike the case of conventionally used electrodes, in which the exposed metallic surface can electrolytically interact with the ionic medium and the metal can induce immune responses, leading to the formation of reactive glial tissues in chronic applications.

(2) Reliability and Stability of Stimulation.

The drive current applied to the microcoil to create magnetic field does not leak out into the ambient tissue, unlike the electrical approach. This improves reliability and stability of the cortical stimulation since the drive current is substantially unaffected by the medium. Notably, the magnetic field generated by the drive current is substantially unaffected by the plastic, dura, or CSF due to a fundamental property of quasi-static magnetic because each of these materials is substantially transparent to the magnetic field at frequencies below about 50 kHz.

(3) Heating of the Tissue and Bone.

As mentioned above, heating of the tissue and overlying bone is potentially a serious problem for electrical stimulation of the spinal cord, but is expected to be significantly reduced or even negligible for magnetic stimulation. In the case of electrical stimulation, the tissue surrounding an a electrode can heat up if there is an exposed edge in the electrode, because electric charges accumulate at edges and produce high densities electric fields. Roughening of the surface of the electrode caused by electrolytic interactions with the ambient medium over time tends, therefore, lead to high concentration and density of electric field and, therefore, heating of the tissue in the vicinity of such roughened regions. The radial component of the electric field formed by an electrode can heat the bone and dura because the radial component of the electric field is reciprocally proportional to the electrical conductivity σ (which is low for both the bone and dura materials), while the longitudinal component of the electric file, which is proportional to σ, may cause the heating of the CSF (that has high electrical conductivity).

In stark contradistinction with the above, in the case of magnetic stimulation of the spinal cord matter proposed in this application, the heating is negligible at least for three reasons.
First, the heating of the coil itself is estimated to be <0.2° C. per pulse;
Second, since the coil is completely encased in a plastic the direct heating of the tissue due to the current in the coil is negligible;
Third, direct tissue heating by the E-vector is also minimal because the E-vector is parallel to the tissues. When the tissues such as the bone and dura are long relative to the length of the coil, as it is the case here, the electric field in each tissue is mostly due to the electric field produced by the coil (E_{coil}). The electric field at the boundary of each region differing in electrical conductivity does not significantly contribute since the E-vector is parallel to the boundary (as shown in FIG. 9). Therefore, the heating effect in any tissue is determined mostly by the E_{coil}. The Joule heating energy W in any given tissue voxel (In reference to FIGS. 9B, 9C) in one cycle of a sinusoidally varying current pulse applied to the coil is: W=2 tᵣσₜᵢₛₛᵤₑVₘEₜᵢₛₛᵤₑ², where tᵣ is the rise time of the time-varying current pulse [s], σₜᵢₛₛᵤₑ is the electrical conductivity of the tissue [S/m], Vm is the volume of the voxel [m³] and Eₜᵢₛₛᵤₑ is the electric field in the voxel [V/m]. The rise of temperature in the bone, ΔT, expressed as a function of the specific heat of the voxel in the bone (estimated to be about 400 J/kgC), the electrical conductivity of the bode (estimated to be about 0.01 S/m), and the voxel bone density (estimated to be about 1,000 kg/m³), and assuming E_{coil} at the surface of the spinal cord to be about 1,000 V/m, can be calculated to be about 3.50 micro-degrees C. per cycle of pulse; and about 350 micro-degrees C. per cycle of pulse in the CSF;

(4) High-Resolution Stimulation and Sharp Focus Activation.

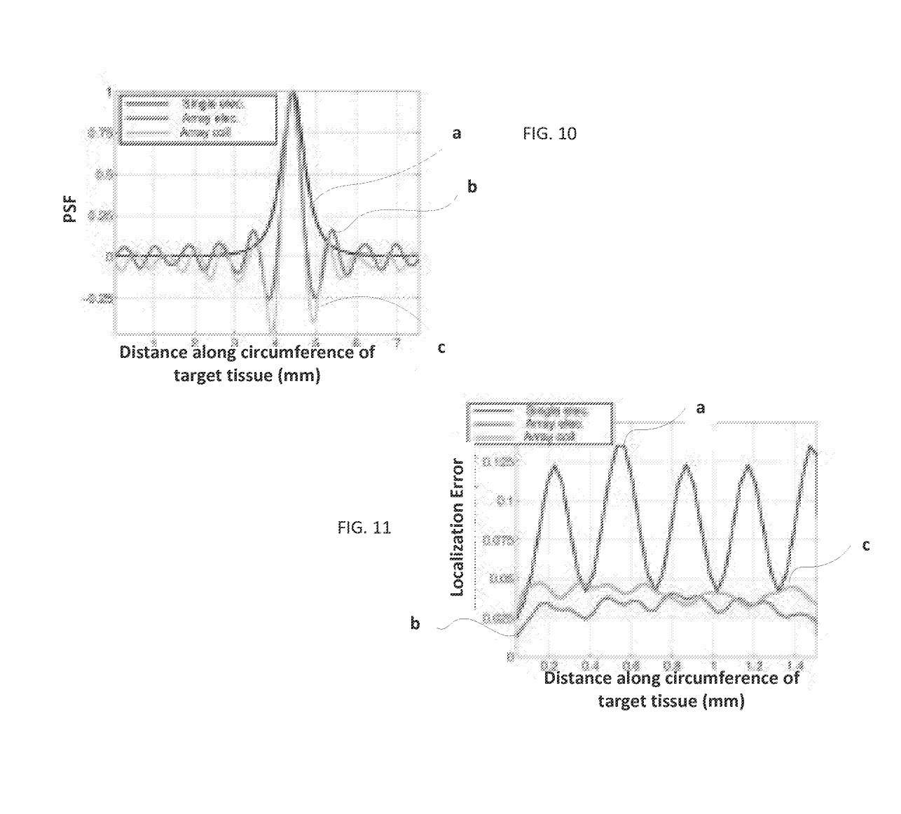

The use of an array of coils provides an additional very crucial advantage to the presented approach. Specifically, as the spinal axons are long in comparison with the coil length (which is about 1.2 mm or so), the boundary effects on the activation function −∂E/dz′ representing the initial rated of change of depolarization of neurons, can be substantially neglected. Representing the axions, in the first-order approximation, without nodes of Ranvier and employing the technique referred to in the field of magnetoencephalography as synthetic aperture stimulation (SAT), the desired target activation can be constructed to be substantially localized. The target axion activation vector t can be expressed as =Xc, where X is a matrix containing the vectors of activation function spatial pattern for a coil as columns and c is the matrix representing the current for different coil channel to be determined. Obtaining the optimal least square estimate for c as C_{LS}=(X^{T}X)⁻¹X^{T} t, and the appropriate scaling the C_{LS} by a value of dI/dt enables a desired localization of the peak of the activation function. To this end, FIG. 10 illustrates the point-spread function of the activation function for a single pair of bipolar electrodes (curve a), and array of coils placed around the spinal cord (see embodiment 200; curve b), and an array of bipolar electrodes place at the two end of each coil in the coil array (in accord with the embodiment 500; curve c), providing evidence that both the embodiment 200 and the embodiment 500 achieve higher degree of localization of the activation of spinal cord neurons.

Moreover, the use of synthetic aperture stimulation in conjunction with the embodiment of the present invention facilitates improvement in targeting of the spinal cord tissue. As attested to by the graphs in FIG. 11, showing (i) the spatial localization error for a single bipolar electrode (curve a) computed by stimulating individual bipolar electrodes in the grid from one part to another as the target was moved along the circumference of a circle at a depth of about 400 microns; (ii) the array of coils (curve b), computed by solving the LS problem for each target location and comparing the location of the peak of the activation function to the actual target location; and (iii) the combination of the array of coils with the grid of bipolar electrodes (curve c). As evidenced by the graphs, the error function corresponding to the embodiments 200, 500 (curves b, c) are substantially smooth in comparison with those corresponding to activation of neurons with electrodes only.

In accordance with examples of embodiment, described with reference to the Figures, a system and method for subdural stimulation of the spinal neurons is provided. The system includes an array of microcoils disposed, in employment, subdurally around the spinal cord. Unlike in a conventionally used approach where the spinal neurons are stimulated with subdural electrodes, with the proposed magnetic approach each coil is completely encased in a biocompatible plastic, thereby eliminating deterioration of the coils, leakage of the current in the coil to the surrounding tissue, and immune reactions between the metal in the coil and tissue. The plastic does not attenuate the magnetic field and eddy current in the spinal cord since the plastic is transparent to the magnetic field below about ˜50 kHz. Moreover, our preliminary study showed that magnetic stimulation with the proposed coil design does not produce the problem of heating in the bon e, in contrast to the electrical approach. The current shunting by the CSF is also less of problem in case of magnetic coils since eddy current produced by each coil is confined to the region of the coil with the current having a mirror-image shape of the current in the coil. In other words, CSF shunts the electric field by its very low conductance generating a virtual “short circuit” on the electrodes. However, CSF cannot shunt the magnetic field, since it is not a magnetic “short circuit” as the magnetic permeability of CSF is the same of the surrounding tissue. Short-circuiting a magnetic field is extremely hard to achieve in practice, since only materials with very high magnetic permeability can perform true magnetic shielding, such as mu-metals which are not present in the human body.

At least some elements of a device of the invention can be controlled, in operation with a processor governed by instructions stored in a memory. The memory may be random access memory (RAM), read-only memory (ROM), flash memory or any other memory, or combination thereof, suitable for storing control software or other instructions and data. Those skilled in the art should also readily appreciate that instructions or programs defining the functions of the present invention may be delivered to a processor in many forms, including, but not limited to, information permanently stored on non-writable storage media (e.g. read-only memory devices within a computer, such as ROM, or devices readable by a computer I/O attachment, such as CD-ROM or DVD disks), information alterably stored on writable storage media (e.g. floppy disks, removable flash memory and hard drives) or information conveyed to a computer through communication media, including wired or wireless computer networks. In addition, while the invention may be embodied in software, the functions necessary to implement the invention may optionally or alternatively be embodied in part or in whole using firmware and/or hardware components, such as combinatorial logic, Application Specific Integrated Circuits (ASICs), Field-Programmable Gate Arrays (FPGAs) or other hardware or some combination of hardware, software and/or firmware components.

References throughout this specification to “one embodiment,” “an embodiment,” “a related embodiment,” or similar language mean that a particular feature, structure, or characteristic described in connection with the referred to “embodiment” is included in at least one embodiment of the present invention. Thus, appearances of the phrases “in one embodiment,” “in an embodiment,” and similar language throughout this specification may, but do not necessarily, all refer to the same embodiment. It is to be understood that no portion of disclosure, taken on its own and in possible connection with a figure, is intended to provide a complete description of all features of the invention.

In addition, when the present disclosure describes features of the invention with reference to corresponding generally-not-to-scale drawings (in which like numbers represent the same or similar elements, wherever possible), the depicted structural elements are generally not to scale, and certain components are enlarged relative to the other components for purposes of emphasis and understanding. It is to be understood that no single drawing is intended to support a complete description of all features of the invention. In other words, a given drawing is generally descriptive of only some, and generally not all, features of the invention. A given drawing and an associated portion of the disclosure containing a description referencing such drawing do not, generally, contain all elements of a particular view or all features that can be presented is this view, at least for purposes of simplifying the given drawing and discussion, and directing the discussion to particular elements that are featured in this drawing. A skilled artisan will recognize that the invention may possibly be practiced without one or more of the specific features, elements, components, structures, details, or characteristics, or with the use of other methods, components, materials, and so forth. Therefore, although a particular detail of an embodiment of the invention may not be necessarily shown in each and every drawing describing such embodiment, the presence of this particular detail in the drawing may be implied unless the context of the description requires otherwise. In other instances, well known structures, details, materials, or operations may be not shown in a given drawing or described in detail to avoid obscuring aspects of an embodiment of the invention that are being discussed. Furthermore, the described single features, structures, or characteristics of the invention may be combined in any suitable manner in one or more further embodiments.

Moreover, if the schematic flow chart diagram is included, the depicted order and steps depicted therein may be indicative of only one embodiment of the presented method, and other steps and methods may be conceived. Without loss of generality, the order in which processing steps or particular methods occur may or may not strictly adhere to the order of the corresponding steps shown.

The invention as recited in claims appended to this disclosure is intended to be assessed in light of the disclosure as a whole, including features disclosed in prior art to which reference is made.

While the invention is described through the above-described examples of embodiments, it will be understood by those of ordinary skill in the art that modifications to, and variations of, the illustrated embodiments may be made without departing from the disclosed inventive concepts. Furthermore, disclosed aspects, or portions of these aspects, may be combined in ways not listed above. Accordingly, the invention should not be viewed as being limited to the disclosed embodiment(s).

What is claimed is:

1. A device for stimulating a biological tissue, the device comprising:
a biocompatible element having a sheet of electrically non-conductive material, first and second surfaces and first and second ends, said element containing a plurality of equal segments extending along an axis between the first and second ends, the immediately neighboring segments adjoining one another along the axis;
microcoil systems juxtaposed with said element to be electrically insulated from the first and second surfaces and to form an array of microcoil systems, which array is oriented transversely to the axis,
wherein each segment of said element is associated with a microcoil system that extends between the first and second ends, and
wherein said element is configured to define a cylindrical surface.

2. A device according to claim 1, further comprising a power coupling configured to connect the microcoil systems to a power source to drive the microcoil systems to produce magnetic fields suitable for performing cortical stimulation.

3. A device according to claim 2, further comprising a stimulator configured to control a delivery of power from the power source to the microcoil systems to deliver an electrical current to a selected combination of the microcoil systems, in a preselected pattern to perform cortical stimulation.

4. A device according to claim 1, further comprising a set of bipolar electrodes equal in number to a number of the microcoil systems, each bipolar electrode disposed along the axis on one of the first and second surfaces such as to overlap with a corresponding microcoil system at ends thereof, the bipolar electrodes and microcoil systems being electrically insulated from each other.

5. A device according to claim 3, wherein a pole of a bipolar electrode from the set includes a circular electrically-conductive patch.

6. A device according to claim 5, wherein the electrically-conductive patch is patterned as a comb.

7. A device according to claim 1, wherein a microcoil system includes a plurality of microcoil elements disposed one over another in layers, wherein first and second immediately neighboring layer are separated from one another by a layer of a microcoil-encasing electrically non-conductive material, each of the microcoil elements having a corresponding via, the microcoil elements being electrically connected to each other through corresponding vias.

8. A device according to claim 7, wherein foot-prints of first and second microcoil elements on the first surface of the implant are co-extensive.

9. A device according to claim 7, wherein the microcoil system further includes a ferrite-based core extending across said layers.

10. A device according to claim 7, wherein the micro-coil-encasing electrically non-conductive material includes biocompatible material.

11. A device according to claim 1, wherein the microcoil systems are evenly spaced in the array of the microcoil systems.

12. A device according to claim 1, further including a biocompatible coating covering at least one of the first and second surfaces, the biocompatible coating forming at least a portion of an electrical insulating barrier that encases the microcoil systems.

13. A device according to claim 1, wherein said element includes a biocompatible implant structured for subdural placement.

14. A spinal cord stimulation device comprising:
a power source operable to produce electric pulses;
an element structured as a sheet containing an array of microcoil systems embedded therein such as to be electrically insulated from ambient medium and structured to receive the electric pulses and generate, in response to said electric pulses, respectively corresponding magnetic fields directed to induce corresponding electrical fields adjacent to the implant sheet and suitable to perform subdural cortical stimulation.

15. A spinal cord stimulation device according to claim 13, wherein said element includes a biocompatible implant sheet.

16. A spinal cord stimulation device according to claim 14, wherein said element is bendable to form a cylindrical surface to direct vectors of said magnetic fields towards an axis corresponding to the cylindrical surface.

17. A spinal cord stimulation device according to claim 14, wherein a microcoil system from the array includes a multilayer structure containing layers with individual microcoils alternating with layers of electrically insulating material, the individual microcoils being electrically connected to each other through vias across the layers of electrically insulating material.

18. A spinal cord stimulation device according to claim 14, further comprising an array of bipolar electrodes overlapped with the array of microcoil systems such that poles of each individual bipolar electrode overlap with external points of a respectively corresponding microcoil system, the bipolar electrodes being operably connected with the power source to receive said electrical pulses.

19. A spinal cord stimulation device according to claim 14, further comprising a controlling circuitry configured to selectively govern the delivery of the electrical pulses to the microcoil systems to effectuate cortical stimulation in a pre-determined spatial pattern.
